(19) United States
(12) Patent Application Publication
(10) Pub. No.: US 2017/0254818 A1
(43) Pub. Date: Sep. 7, 2017

(54) CNS-SPECIFIC BIOMARKERS FOR CNS DISEASES OR INJURIES

(71) Applicants: William E. HASKINS, San Antonio, TX (US); Thomas G. FORSTHUBER, San Antonio, TX (US)

(72) Inventors: William E. HASKINS, San Antonio, TX (US); Thomas G. FORSTHUBER, San Antonio, TX (US)

(73) Assignee: THE BOARD OF REGENTS OF THE UNIVERSITY OF TEXAS SY STEM, Austin, TX (US)

(21) Appl. No.: 15/506,325

(22) PCT Filed: Aug. 24, 2015
(86) PCT No.: PCT/US15/46598

§ 371 Date: Feb. 24, 2017

Related U.S. Application Data
(60) Provisional application No. 62/041,099, filed on Aug. 24, 2014.
(60) Provisional application No. 62/044,501, filed on Sep. 2, 2014.

(51) Int. Cl. G01N 33/68

(52) U.S. Cl. CPC G01N 33/6896; G01N 2800/285

(57) ABSTRACT

Methods and compositions for detecting and/or measuring biomarkers associated with compromised BBB.

This Application claims priority to U.S. Provisional Patent Applications 62/041,099 filed Aug. 24, 2014, and 62/044,501 filed Sep. 2, 2014, each of which are incorporated herein by reference in their entirety.

### STATEMENT REGARDING FEDERALLY FUNDED RESEARCH

This invention was supported by National Institute of Health grants G12MD007591, grants NS52177 and NS084201, NIH5U54RR022762-05. The government has certain rights in this invention.

### FIELD OF THE INVENTION

The present invention relates generally to the fields of neurology, immunology, and diagnostics. In particular, the present invention relates to the identification and detection of biomarkers in biological samples indicative of neuronal and central nervous system disease.

### BACKGROUND

Multiple sclerosis (MS) is a debilitating disease of the central nervous system (CNS) that affects approximately 2.5 million people globally (Mateen et al. Lancet Neurol 11: 484-485, 2012). MS patients experience episodes of inflammation and demyelination that are believed to be mediated by an autoimmune attack directed against CNS-specific proteins (CSPs) of the myelin sheath on axons, including: myelin basic protein, proteolipid protein, and myelin oligodendrocyte glycoprotein. The autoimmune attack promotes an inflammatory cascade in the CNS highlighted by recruitment of innate- and adaptive-immune cells and release of inflammatory mediators that act in concert to damage the myelin sheath and neuronal axons.

Autoreactive T cells have been shown to be key mediators of this attack against the myelin sheath. Once activated, these T cells migrate and infiltrate into the CNS, crossing the blood-brain-barrier (BBB) in a multistep process (Steinman, Cell 85, 299-302, 1996; Goverman Nat Rev Immunol 9, 393-407, 2009). Infiltrating autoreactive T cells release inflammatory cytokines that modulate the activation of microglia, infiltrating macrophages, and dendritic cells to release neurotoxic mediators, including nitric oxide and reactive-oxygen species (ROS) (Rovaris et al. Lancet Neurol 5, 343-354, 2006; Dhib-Jalbut et al. Journal of neuroimmunology 176, 198-215, 2006). Macrophages, microglia, and dendritic cells are also actively involved in the inflammatory response (Hartung et al. J Neuroimmunol 40, 197-210, 1992; Benveniste, J Mol Med (Berl) 75, 165-173, 1997). While MS research has historically focused on inflammatory events in the CNS, such as the pathological role of cytokines (Veldhoen, Current opinion in immunology 21, 606-611, 2009; Olsson, Journal of neuroimmunology 40, 211-218, 1992), a more detailed molecular understanding of the biology of other proteins, particularly CSPs transported across the blood brain barrier (BBB) into cerebrospinal fluid (CSF) or serum, is critical to further the understanding of damage to the BBB and to develop new biomarkers and treatments based on this understanding.

There is a need for additional methods for assessing central nervous system integrity and diseases.

### SUMMARY OF THE INVENTION

Detection or profiling of central nervous system-specific proteins (CSPs) traversing a compromised or damaged blood-brain-barrier (BBB) to cerebrospinal fluid (CSF) and/or blood (e.g., serum) is a promising diagnostic, prognostic, and/or predictive method. Certain embodiments are directed to methods of identifying and/or detecting/quantitating CSP proteins or peptides in the CSF or serum. In certain aspects the protein biomarker(s) are not detected at appreciable levels in the circulation of healthy subjects. In further embodiments a microwave and magnetic (M2) proteomics assessment of CSPs in brain tissue is used to detect and/or prioritize CSP biomarkers in serum or for producing serum profiles. In certain aspects the biomarkers are detected using immunoassays. In a further aspect the biomarkers are measured over time to provide a biomarker signature.

In certain aspects a mouse model of multiple sclerosis is used as a representative model of BBB compromise. M2 proteomics is used to assess CSP expression in brain tissue or CSP presence in blood from mice during induction of experimental autoimmune encephalomyelitis (EAE). Confirmation of CNS-infiltrating inflammatory cell response and CSP presence or expression in serum is achieved with cytokine ELISPOT and ELISA immunoassays. M2 proteomics (and ELISA) revealed characteristic CSP expression waves, including synapsin-1 and α-II-spectrin, which peaked at day 7 in brain tissue (and serum) and preceded clinical EAE symptoms that began at day 10 and peaked at day 20. As used herein an expression wave is a change in expression (increase, decrease, or increase and decrease) of one or more proteins over a time period. Expression trajectory is used to refer to the rate of change (slope of the expression curve) of expression over time, e.g., expression that is increasing has a positive trajectory or slope whereas expression levels that are decreasing have a negative trajectory or slope. In certain aspects expression levels will be maintained giving rise to a flat trajectory or a slope of about 0. In certain aspects an expression wave of one or more biomarkers precedes a clinically detectable or definable event, and thus can be used as a predictive tool, e.g., it is a preclinical or pre-symptom indicator of a disease state. Various biomarkers may be detected in a number of expression waves that may be synchronized or out of phase with each other. In certain aspects one or more expression waves are correlated with a current or future condition or progression of the subject and can be used as a diagnostic, prognostic, or predictive tool. In one example an expression wave detected in an asymptomatic subject can diagnose a subject with a CNS disease or condition.

Embodiments are directed to methods and compositions for detecting one or more biomarkers or assessing a panel of biomarkers (profile) or generating a biomarker signature (expression profile over time—expression wave). As used herein a profile refers to the levels of a plurality of biomarkers at a particular time, whereas a biomarker signature or expression wave refers to a plurality of biomarker levels over a period of time, as indicated by obtaining and analyzing samples at multiple times. In certain aspects the period of time is sampled over 2, 3, 4, 5, 6, 7, 8, 9, 10 or more time points. In a further embodiment the time points are at least, about, or at most 1, 2, 3, 4, 5, 6, 7, 8, 9, 10, 11, 12, 20, 24, 30, 40, 50, or 60 hours or days apart. A biomarker signature or several biomarker levels at different time points can be used to characterize an expression wave of biomarkers. The biomarker signature can be assessed and used to classify a subject during treatment in order to characterize the progression, suppression, or regression of symptoms or a disease state in a patient over time. The biomarker signature can be used to assess the effectiveness of treatments and allow the modification of treatments or use of alternative treatments tailored to a particular patient having a particular biomarker profile or signature. Such assessment and modification of treatments can be used to yield a more favorable patient outcome. In certain aspects a biomarker profile is produced at various time points before, during, and/or after a treatment is administered. In certain aspects, blood samples are collected every 1, 2, 3, 4, 5, 6, 7, or more days, weeks, or months.

In certain embodiments a biological sample is contacted with a label or detection reagent that forms a conjugate or complex with a biomarker described herein. The complex is then detected by analyzing the labeled biomarker or detecting the biomarker/detection reagent complex. In certain aspects biological samples can be taken at two separate time points from the same subject or patient. In a further aspect the levels of the biomarker(s) are determined relative to a reference, control, or relative to time points at which samples are taken. In certain rate of change across a plurality of samples taken at different time points is determined.

In certain aspects the patient is suspected of having, diagnosed as having, or is at risk of having a compromised BBB or a condition or disease related to or resulting in a compromised BBB. A subject can be suspected of having a condition based on physiologic tests or symptomatic occurrences. A subject may be asymptomatic but at risk if the subject has a family history of a condition, a genetic biomarker indicating a probability of developing a condition, or the subject has been exposed to an environment, agent, or event that is known to cause or increase the occurrence of a condition. In a further aspect the patient is diagnosed with a neurodegenerative disease or a brain injury.

Certain embodiments are directed to prognostic or diagnostic methods for MS or similar conditions. In certain aspects the methods can be used to characterize or stage MS using the disclosed biomarkers and/or biomarker signatures. In a further aspect, one can adjust or modify the treatment of a patient based on the characterization or stage of disease. In certain aspects the modification or adjustment of a treatment includes modifying the dosage, intensity, length of treatment, frequency of treatment, and inclusion or elimination of any additional drug or combination of drug treatments, e.g., beta-interferons, therapeutic antibodies, Tysabri, glucocorticoids, natalizumab, fingolimod and/or glatiramer acetate.

In certain aspects the characterization of a subject or patient over time in conjunction with treatments can be used to identify treatments and therapies that ameliorate or prevent progressive episodes of neurodegenerative condition. In other aspects, toxic effects of treatments can be minimized by monitoring the biomarker profile or signature, and reducing or stopping treatments once a progression event has lessened or passed. In certain aspects a treatment is stopped once a biomarker signature indicates an end or a lessening of a progression event. In a further aspect a treatment is administered when a biomarker signature indicates the initiation or worsening of a progression event.

Certain embodiments are directed to methods for diagnosing a neurological disease in a subject associated with a compromised BBB. In certain aspects the method comprises determining an amount or detecting the presence of at least one peptide having an amino acid sequence of SEQ ID NO:1 to SEQ ID NO:544 in a biological sample from the subject. In certain aspect at least 2, 5, 10, 20, 40, 80, 160, 200, 300, 400, 500 or more peptides corresponding to SEQ ID NO:1 to 544 are measured in sample obtained at 1, 2, 3, 4, 5, 6, 7, 8, 9, 10 or more time points. In certain aspects the time point samples are obtained at least, about, or at most 1, 2, 3, 4, 5, 6, 7, 8, 9, 10, 11, 12, 13, 14, 15, 16, 17, 18, 19, 20, 21, 22, 23, 24, hours, days, or weeks apart. The amount, profile, or signature of one or more of the peptide(s) in the sample can be compared to a control level or a reference, wherein if the biomarker levels in the sample are different than the control level or reference, the subject can be diagnosed as having, or at an increased risk of developing, the neurological disease. In certain aspects aberration from a normal biomarker profile is indicative of degeneration of the BBB. Certain aspects are directed to a composition comprising a biological sample isolated from a subject and one or more antibodies that specifically and independently bind a peptide having an amino acid sequence of SEQ ID NO:1 to SEQ ID NO:544. In other aspects a peptide having an amino acid sequence of SEQ ID NO:1 to SEQ ID NO:544 can independently and separately form a complex with a detection reagent.

In certain aspects one or more peptide is selected from SEQ ID NO:465, SEQ ID NO:499, SEQ ID NO:265, SEQ ID NO:341, SEQ ID NO:384, SEQ ID NO:475, SEQ ID NO:503, SEQ ID NO:235, SEQ ID NO:383, SEQ ID NO:434, SEQ ID NO:544, and/or SEQ ID NO:163.

In a further aspect one or more peptide is selected from SEQ ID NO:39, SEQ ID NO:544, SEQ ID NO:163, SEQ ID NO:235, SEQ ID NO:265, SEQ ID NO:341, SEQ ID NO:375, SEQ ID NO:377, SEQ ID NO:378, SEQ ID NO:383, SEQ ID NO:384, SEQ ID NO:434, SEQ ID NO:475, SEQ ID NO:499, and/or SEQ ID NO:503.

In certain aspects the method comprises determining an amount or detecting the presence of at least one protein having an amino acid sequence of SEQ ID NO:545 to SEQ ID NO:923, or the human homolog thereof, in a biological sample from the subject. In certain aspects at least 2, 5, 10, 20, 40, 80, 160, 200, 300 or more proteins corresponding to one or more of SEQ ID NO:545 to 923, or human homolog, are measured in sample obtained at 1, 2, 3, 4, 5, 6, 7, 8, 9, 10 or more time points. The amount, profile, or signature of one or more of the peptide(s) in the sample can be compared to an earlier and/or later time point(s), a control level or a reference, wherein if the biomarker levels in the sample are different than the control level or reference, the subject can be diagnosed as having, or at an increased risk of developing, the neurological disease. In certain aspect aberration from a normal biomarker profile is indicative of degeneration of the BBB. Certain aspects are directed to a composition comprising a biological sample isolated from a subject and one or more antibodies that specifically and independently bind a protein having an amino acid sequence of SEQ ID NO:545 to SEQ ID NO:923, or the human homolog.

In certain embodiments, a biomarker can be 1, 2, 3, 4, 5, 6, 7, 8, 9, 10, 11, 12, 13, 14, 15, 16, 17, or more of α-II-spectrin, synapsin-1/2/3, myelin basic protein, ubiquitin carboxyl-terminal esterase L1, calcium/calmodulin-dependent protein kinase II alpha, neurofilament light and medium, parkin 2, peroxiredoxin-1/4/5/6, 14-3-3-β/ε/ζ, synaptotagmin, enolase-1/2, guanine nucleotide binding protein α and β1, superoxide dismutase 2, internexin neuronal intermediate filament protein α, tyrosine-protein phosphatase non-receptor type substrate 1, macrophage migration inhibitory factor, proteolipid protein 1, caspase 7, or combinations thereof.

In other embodiments one or more biomarkers can be selected from Neural cell adhesion molecule L1-like protein (CHL1), Neurofilament Medium (NF-M), Synaptotagmin 1 (SYT), Synapsin 3 (Syn 3), Spectrin alpha chain, brain (SPTAN1), Calcium/calmodulin-dependent protein kinase type II alpha chain (CAMK2A), Neurofilament Heavy (NF-H), Enolase 1 (ENO1), Synapsin 2 (Syn 2), Phosphatidylethanolamine binding protein 1 (PEBP1), Neurofilament Light (NF-L), Parkin (Park2), Enolase 2 (ENO2), Synapsin I (Syn 1), or combinations thereof.

Certain embodiments are directed to methods of evaluating a patient suspected of having a CNS disease comprising measuring the level of at least one protein and/or peptide found in a biological sample from a patient presenting with clinical symptoms consistent with a CNS disease or is suspected as having or being at risk of developing such disease, and diagnosing or evaluating the patient based on the levels of one or more biomarker in one or more biological sample. In certain aspects the one or more biological samples are taken over a period of days, weeks, or months. In certain aspects the presence or absence of one or more protein and/or peptide associated with a CNS disease or an exacerbation of a CNS disease indicates the diagnosis or the likelihood of developing a particular disease or classifies the severity or stage of a disease.

In certain aspects the biomarkers are used to produce a biomarker signature comprising a plurality of biomarkers measured over time. In a further aspect the biomarkers are evaluated at 2, 3, 4, 5, or more time points. The time point being 1, 2, 3, 4, 5, 6, 7, or more days, weeks, or months apart. The biomarkers are indicative of disease, disease progression, or disease severity. Patients with biomarker levels indicative of such disease can be diagnosed with a CNS disease or characterized as to the severity of a CNS disease.

The methods may further comprise administering a neuroprotective treatment to the patient based on a biomarker profile or the level of one or more proteins or peptides, even if no clinical symptoms are evident. In a further aspect the biomarker(s) or biomarker profile is used to monitor the response to treatment and/or disease progression.

In some embodiments, the levels of a plurality of proteins and/or peptides in the patient's sample can be used to prepare a biomarker profile or signature associated with CNS disease. The levels of proteins and/or peptides in the profile may be determined relative to levels of the proteins and/or peptides in control samples or normal samples or pre-determined reference values or ranges of reference values. The biomarker profile is, in some embodiments, indicative of the presence, stage, or severity of CNS disease in a patient. When such biomarker profiles or signatures are prepared from samples obtained from patients following administration of a neuroprotective treatment, the biomarker profile or signature, or changes in biomarker profile or signature can be used as an indicator of therapeutic efficacy of the neuroprotective treatment in the patient. These biomarker profiles can be used to monitor the progression or regression of a patient being treated. In some embodiments the neuroprotective treatment will be an experimental treatment, a glucocorticoid treatment, a beta-interferon treatment, a therapeutic antibody treatment, a Tysabri treatment, a natalizumab treatment, a fingolimod treatment, a glatiramer acetate treatment, or any combination thereof.

Certain embodiments are directed to methods for determining treatment efficacy and/or progression of a neurological disease in a subject. In some embodiments, the method comprises determining an amount of at least 1, 2, 3, 4, 5, 6, 7, 8, 9, 10, 11, 12, 13, 14, 15, 16, 17, 18, 19, 20 or more biomarkers described herein, in a first biological sample collected from the subject at a first time point; determining an amount of the selected biomarkers in a second biological sample from the subject at a second time point; and comparing the amounts of the selected biomarkers in the first and second samples, wherein a change in the amounts of the biomarkers from the first and second samples is indicative of treatment efficacy and/or progress of the neurological disease in the subject. In certain aspects, the first time point is prior to initiation of a treatment for the neurological disease and the second time point is after initiation of the treatment. In other embodiments, the first time point is prior to onset of the neurological disease and the second time point is after onset of the neurological disease. Determining an amount of a biomarker can comprise contacting the sample with a detection reagent that forms a complex with a biomarker and then detecting the complex. In certain aspects the detection reagent is an antibody or a label. In certain aspects the complex is detected by ELISA or mass spectrometry.

In certain aspects the subject or patient is human. In a further aspect the subject is at risk of developing, suspected of having, or has a condition associated with a compromised BBB. In certain aspect the subject is a multiple sclerosis patient. In a further aspect, the multiple sclerosis patient has relapsing remitting multiple sclerosis, a progressive multiple sclerosis, or both.

The present invention also provides a method of determining a prognosis for a patient with a central nervous system disease. In one embodiment, the method comprises detecting and/or quantitating in a biological sample one or more biomarkers in the biological sample and generating a biomarker profile or signature, wherein the biomarker profile or signature is indicative of the prognosis of the patient. In certain aspects the biological sample is cerebrospinal fluid or blood, or a component thereof, e.g., serum, plasma, etc. In particular embodiments, an elevated level of at least one protein and/or peptide relative to a pre-determined reference value or range of reference values is indicative of disease progression. For, example elevated synapsin-1, or fragments thereof, levels are predictive of the patient having a relapse of their disease.

In certain aspects a biological sample is obtain from a patient who recently (within the preceding 1, 2, 3, 4, or more days, weeks, or months) experienced an initial clinical attack. In certain aspects the sample is obtained prior to administration of treatment. In a further aspect samples are taken at various time points after the initial clinical attack to monitor treatment efficacy and disease course. Samples can be obtained periodically whether clinical symptoms persist or not. In certain aspects a sample is obtained about every 1, 2, 3, 4, 5, months or so. In certain aspects the biomarker profile or signature is correlated with (a) disease remission and therapeutic efficacy including proper dosing of therapies, (b) success or failure of therapy, (c) disease stage, e.g., is a patient progressing, (d) expression waves that are indicative of relapse, etc.

Upon producing the biomarker signature or measuring levels of a protein and/or peptide selected from the biomarker listed herein a physician would then determine: whether the disease is in remission and the drugs are working, whether the drugs are failing or have failed, whether the patient is progressing, and/or whether a patient is at risk of relapse.

Certain embodiments include kits for use in generating biomarker profiles or signatures, or measuring one or more biomarkers associated with a disease. In one embodiment, the kit comprises a probe, such as labeled-antibody or fragment thereof, that labels or specifically binds to biomarker described herein, including the peptides or proteins in SEQ ID NO:1-923. In certain aspects the biomarker is selected from human or mouse α-II-spectrin, synapsin-1/2/3, myelin basic protein, ubiquitin carboxyl-terminal esterase L1, calcium/calmodulin-dependent protein kinase II alpha, neurofilament light and medium, parkin 2, peroxiredoxin-1/4/5/6, 14-3-3-β/ε/ζ, synaptotagmin, enolase-1/2, guanine nucleotide binding protein α and β1, superoxide dismutase 2, internexin neuronal intermediate filament protein α, tyrosine-protein phosphatase non-receptor type substrate 1, macrophage migration inhibitory factor, proteolipid protein 1, and/or caspase 7.

In certain embodiments, a biomarker can be measured and/or detected by mass spectrometry (MS) analysis (e.g., matrix-assisted laser desorption/ionization (MALDI) time-of-flight (TOF) MS analysis, Microwave and Magnetic (M2), or electrospray ionization ((ESI) MS), immunoassay analysis (e.g., enzyme-linked immunosorbent assay (ELISA)), multiplexed bead assays or combinations thereof.

In certain embodiments a sample can be fractionated or modified before detecting or measuring biomarkers. In certain aspects abundant proteins are removed from the sample prior to detecting or measuring a target biomarker. For example albumin and/or immunoglobulin are removed from the sample prior to detection or measurement.

A biological sample includes all or a portion of a saliva sample, a blood sample, a serum sample, a plasma sample, a urine sample, or a cerebrospinal fluid (CSF) sample. In certain aspect the biological sample is a blood or CSF sample.

In certain aspects a patient can be monitored using a biomarker panel described herein and provided a treatment regime personalized for that patient based on the biomarker signature or biomarker profile of that patient.

In certain embodiments treatments are monitored and optimized by: detecting a biomarker signature in biological samples from an individual comprising determining expression levels for 2, 3, 4, 5, 6, 7, 8, 9, 10, 11, 12, 13, 14, 15, 16, 17, 18, 19, 20 or more biomarkers described herein; and determining or identifying a treatment regimen and/or treatment initiation point based on the biomarker signature. In certain aspects treatment initiation is based on the clinical responsiveness to therapies correlated with particular biomarker signatures, e.g., symptoms that follow a particular expression wave.

In other aspects, a likelihood of the onset, or clinical progression of symptoms in an individual can be determined by measuring or detecting one or more biomarker over time (biomarker signature), and comparing the biomarker signature with a reference or standard biomarker signature associated with clinical symptoms, the potential for manifestation of clinical symptoms, and/or treatment conditions, and determining or identifying an individual that has or is at risk of having a disease (e.g., multiple sclerosis) and/or characterizing the stage of the disease. In certain aspects the biomarker signature will indicate an increased level or an increasing level of biomarker relative to a reference or normal biomarker signatures. In certain aspect the reference or normal biomarker signature is that of a healthy individual(s), an individual without any clinical symptoms of a CNS disease, and/or patients with a CNS disease that have been characterized at a particular disease milestone or stage.

Certain aspects employ a step of correlating the presence or amount of biomarker described herein in a biological sample with the severity, stage, or presence of a CNS disease or condition. The amount or time course of one or more biomarker in the biological sample directly relates to severity, diagnosis, prognosis, or predictability of a CNS disease or condition which in turn causes a larger amount of biomarker(s) to accumulate in or change over time in the biological sample (e.g., serum). The results of such a test can help a physician determine whether the administration, alteration, or cessation of a therapy might be of benefit to a patient.

A “biological sample” as used herein refers to a sample of biological tissue or biological fluid. Examples of biological samples are sections of tissues, blood, blood fractions, plasma, serum, urine, CSF, or samples from other sources. A biological sample may be provided by removing a sample of cells or a portion of fluid from a subject, but can also be provided by using a previously isolated sample. For example, a tissue sample can be removed from a subject suspected of having a disease by conventional biopsy techniques. In certain embodiments, a blood sample is taken from the subject. In one embodiment, the blood or tissue sample is obtained from the subject prior to initiation of and/or during treatment.

“Polypeptide”, “protein”, or “peptide” refers to polymer of amino acids joined by peptide bonds or modified peptide bonds. “Polypeptide” refers to longer chains, including proteins, whereas peptide refers to shorter chains of 100 amino acids or less.

“Antibody” refers to all isotypes of immunoglobulins (IgG, IgA, IgE, IgM, IgD, and IgY) including various monomeric and polymeric forms of each isotype, unless otherwise specified. “Functional fragments” of such antibodies comprise portions of intact antibodies that retain antigen-binding specificity of the parent antibody molecule.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The antibodies or functional fragments thereof can be labeled or otherwise conjugated to various chemical or biomolecule moieties, for example, for diagnostic or detection applications. The moieties can be detectable labels, for example, fluorescent labels, radiolabels, biotin, and the like, which are known in the art. There are a wide variety of fluorophore labels that can be attached to the antibodies and probes. Common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCPCy5.5, PE-Cy5, PE-Cy5.5, PE-Cy7, PE-Texas Red, and APC-Cy7. Other fluorophores include, inter alia, Alexa Fluor® 350, Alexa Fluor® 488, Alexa 25 Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling antibodies or other biomarker probes. For secondary detection using labeled avidin, streptavidin, captavidin or neutravidin, the antibodies can be labeled with biotin. When antibodies are used, e.g., for western blotting applications, they can usefully be labeled with radioisotopes, such as ³³P, ³²P, ³⁵S, ³H, and ¹²⁵I.

The terms “treating” or “treatment” refer to any attempt to attenuate or ameliorate an injury, pathology or condition, including any objective or subjective parameter such as abatement, remission, diminishing of symptoms, pathology, or making a condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

As used herein, the term “subject” refers to any mammal, including both human and other mammals. Preferably, the methods of the present invention are applied to human subjects.

“Prognosis” refers to a prediction of how a patient will progress, and whether there is a chance of recovery. A good or bad prognosis may, for example, be assessed in terms of patient survival, likelihood of disease recurrence, or disease progression (which is assessed in relation to a defined time point).

In one embodiment, the biomarker level is compared to a reference level representing the same biomarker in a non-disease context. In certain aspects, the reference level may be a reference level of expression from non-diseased sample(s) from a subject(s) not diagnosed with the disease in question. Alternatively, reference level may be a reference level of expression from a group of subjects. The reference level may be a single value or may be a range of values. The reference level of expression can be determined using any method known to those of ordinary skill in the art. In some embodiments, the reference level is an average level of expression determined from a cohort of subjects. The reference level may also be depicted graphically as an area on a graph. The reference level may comprise data obtained at the same time (e.g., in the same hybridization experiment) as the patient's individual data, or may be a stored value or set of values e.g. stored on a computer, or on computer-readable media. If the latter is used, new patient data for the selected marker(s), obtained from initial or follow-up samples, can be compared to the stored data for the same marker(s) without the need for additional control experiments.

The phrase “specifically binds” or “specifically immunoreactive” to a target refers to a binding reaction that is determinative of the presence of the molecule in the presence of a heterogeneous population of other biologics. Thus, under designated conditions, a specified molecule or probe (e.g., antibody) binds preferentially to a particular target and does not bind in a significant amount to other biologics present in the sample. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following descriptions form part of the present specification and are included to further demonstrate certain aspects of the present invention. The invention may be better understood by reference to one or more of these drawings in combination with the detailed description of the specification embodiments presented herein.

FIG. 1. Disease trajectory for the entire cohort studied herein (top), where mice were evaluated daily for EAE clinical symptoms. The disease distribution and disease trajectory for the mice for M² proteomics are also shown (bottom and inset, respectively). Mice for M² proteomics were randomly selected and sacrificed from 8 disease time points (18≦n≦20 specimens per time point), described by the number of days (d) post-immunization [−1 d (non-immunized), 0 d (3hrs post-immunization), 5 d, 7 d, 10 d, 15 d, 20 d and 25 d]. These time points were selected to reflect inflection points of pre-onset, disease onset, early disease, disease peak and remission.

FIG. 2. M² proteomics trajectories for selected differentially expressed peptides from synapsin-1 (A2AE14_MOUSE). Values for (1) significant differential expression (AUC>0.9) between two post-immunization time points and (2) significant differential expression (overall p-value<1.0E-03) across all post-immunization time points are provided in Supplementary Information.

FIG. 3. Synapsin-1 (A2AE14_MOUSE) yielded (A) 5 peptides with significant differential expression (pair-wise time point contrasts) between 5 vs. 7 days and (B) 5 peptides with significant differential expression between 0 vs. 7 days post-immunization.

FIG. 4. ELISA results for levels of two CSPs: (A) synapsin-1 in brain tissue, (B) α-II-spectrin in brain tissue, and (C) synapsin-1 (gray) and α-II-spectrin (black) in serum.

FIG. 5. ELISPOT results for levels of two pro-inflammatory cytokines: (A) IFN-γ in spleen tissue, (B) IL-17 in spleen tissue, (C) IFN-γ in brain tissue, and (D) IL-17 in brain tissue.

FIG. 6. Hierarchical clustering of risk groups by correlating relative peptide expression to post-immunization time for a subset of CSPs and other putative protein biomarkers measured by M² proteomics. (A) shows non-supervised clustering for all CSPs, while (B) shows representative supervised hierarchical clustering results for synapsin-1 (A2AE14_MOUSE).

FIG. 7. Time-specific analysis of the CNS-proteome during EAE reveals expression −waves.

FIG. 8. Proteomics analyses of CNS in the pre-onset phase reveals a list of potential biomarkers to predict disease onset.

FIG. 9. Prioritization of biomarkers by bioinformatics and expression trajectories.

FIG. 10. Serum levels of biomarkers predict onset and correlate with CNS proteome changes.

FIG. 11. Serum levels of biomarkers predict onset and correlated with CNS proteome changes.

FIG. 12. Serum levels of biomarkers predict onset and correlated with CNS proteome changes.

### DETAILED DESCRIPTION OF THE INVENTION

Multiple sclerosis (MS) can be used as one example of a disease state that involves the compromise of the blood brain barrier (BBB). Clinical diagnosis of MS, versus other similar neurological diseases, and classification into the consensus definitions of the four major subtypes of MS is based on a limited diagnostic repertoire, including: clinical appearance, disease history and laboratory imaging and/or diagnostics (Steinman, Cell 85, 299-302, 1996; Sospedra and Martin, Annu Rev Immunol 23, 683-747, 2005; Noseworthy et al., N Engl J Med 343, 938-952, 2000; Thompson et al., Brain: a journal of neurology 120 (6), 1085-1096, 1997; Lublin and Reingold, Neurology 46, 907-911, 1996; Confavreux et al., The New England journal of medicine 343, 1430-1438, 2000). Currently, MS is classified into relapsing-remitting (RRMS), secondary-progressive (SPMS), primarily-progressive (PPMS) and progressive-relapsing (PRMS). Approximately 80% of the patients initially develop the RRMS form of the disease, characterized by clinical attacks (relapses) with diverse neurological dysfunctions, followed by functional recovery (remission). More than half of these patients will eventually develop SPMS, characterized by progressive residual neurological deficiencies with or without attacks during the progressive phase (Lublin and Reingold, Neurology 46, 907-911, 1996). Current immunomodulatory treatments ameliorate, but do not cure, MS, including: beta-interferons, therapeutic antibodies, glucocorticoids, and glatiramer acetate. Responses to treatments are highly variable between patients and no accurate means exist to predict efficacy of a particular drug. Individual responses to treatment are typically evaluated by clinical measures of disease progression such as the expanded disability status scale (EDSS) (Poonawalla et al., Mult Scler 16, 1117-1125, 2010) and magnetic resonance imaging (MRI) of brain lesion volume (Bakshi et al., Lancet Neurol 7, 615-625, 2008; Tourdias and Dousset, Neurotherapeutics, 2012; Filippi and Agosta, J Magn Reson Imaging 31, 770-788, 2010; Neema et al., Neurotherapeutics 4, 602-617, 2007). However, these clinical measures lack sensitivity and specificity for a large population of MS patients, and they fail to show a strong correlation between specific treatments and their efficacy in slowing disease progression in individual MS patients.

Thus, there is a need for molecular biochemical markers (biomarkers) with improved diagnostic, prognostic, and predictive power. However, poorly understood variations of genetic, environmental, and socioeconomic factors in the MS patient population present profound challenges for biomarker research. A diagnostic matrix with a particular combination of biomarkers might enable more precise molecular stratification of individual patients into treatment groups. Moreover, a particular combination of biomarkers might be necessary because not all of these molecules are expected to be exclusive to MS and might also be found in other diseases and neurological disorders.

Studying the relation of protein expression trajectories to disease progression within individual MS patients can mitigate population variability to a certain degree and account for potential patient-specific factors. More than 24,000 genes are translated and post-translationally modified into an estimated 2 million protein isoforms in humans, encoding far more molecular diversity than the relatively static genome or transcriptome. Paradoxically, less than 100 proteins are routinely quantified in serum today (Rifai et al., Nat Biotechnol 24, 971-983, 2006; Anderson and Anderson, Mol Cell Proteomics 1, 845-867, 2002). Thus, the most sensitive (most true-positive) and specific (least false-positive) biomarkers are expected to be at the protein level. Notably, proteins must be measured directly due to the poor correlation between the transcriptome and proteome due to alternative splicing, post-translational modifications, single nucleotide polymorphisms, limiting ribosomes available for translation, mRNA, protein stability and other factors (e.g., microRNA).

CNS specific proteins (CSPs), transported across the damaged BBB to cerebral spinal fluid (CSF) and/or blood are promising diagnostic, prognostic, and predictive (therapeutic) protein biomarkers of disease in individual MS patients because they are not expected to be present at appreciable levels in the circulation of healthy subjects. Compared to the highly variable clinical spectrum of individual MS patients, disease in groups of mice with experimental autoimmune encephalomyelitis (EAE), the major animal model for MS, is less heterogeneous and more synchronous, providing a strong rationale for preclinical biomarker studies.

### I. BIOMARKERS

A biomarker is an organic biomolecule, such as a protein or fragment thereof, that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. As such, they are useful as markers for disease (diagnostics), therapeutic effectiveness of a drug (theranostics) and of drug toxicity.

Aspects described herein seek to develop methods for identifying, monitoring, and classifying patients at risk of having a compromised BBB based expression profiling. However, identification of predictive biomarkers in complex biofluids, such as plasma, have been challenging for proteomics technologies. Plasma is a complex biofluid, with its constituent proteins present in a broad dynamic concentration range spanning 6 log orders of magnitude or more (Anderson and Anderson Mol Cell Proteomics 1:845-67, 2002; Rifai and Gerszten, Clinical Chemistry 52:1635-37, 2006). Moreover, high-abundance proteins tend to adsorb lower-abundance proteins and peptides (Gundry et al. Proteomics Clin. Appl. 1:73-88, 2007; Seferovic et al., J Chrom. B Analyt. Technol. Biomed. Life Sci. 865:147-152, 2008). The presence of proteases may produce peptide fragments (Villanueva et al., J Proteome Res. 4:1060-72, 2005; Villanueva et al., Mol. Cell Proteomics 7:509-18, 2008), and the individual variation in plasma protein abundances serve to compound the difficulties in comprehensive proteomic analyses of plasma. With the advancement of multidimensional profiling techniques, the systematic and quick identification of predictive proteins associated with a disease is now feasible.

The spectrin-family is comprised of a group of membrane-bound proteins, including α-II-spectrin, that are present in most vertebrate tissues and were initially discovered as a component of erythrocyte membrane. Spectrin is composed of two subunits, α and β, that coil around each other to form a heterodimer. The α subunit is encoded by two different genes, while the β subunit is encoded by five different genes: alternative splicing generates additional isoforms. In the CNS, almost all major cell types express spectrin, with distinct isoforms found in different cell types. α-II-spectrin is predominantly localized to axons and to presynaptic terminals of neurons with an essential role in Ca+ mediated exocytosis and neurotransmitter release through its association with synapsin proteins that are found in the neuronal vesicles. α-II-spectrin is also cleaved by calcium-dependent cysteine proteases such as calpains and caspases during necrosis and apoptosis to generate breakdown products that are often protease-specific.

Synapsin-1, one of the CSPs reported in this application, is a phosphorylated CSP found at synaptic vesicles in neurons that can bind to several cytoskeleton components including actin, microtubules and α-II-spectrin. It is involved in synaptogenesis and calcium-dependent neurotransmitter release from synaptic vesicles, particularly glutamate release.

Biomarkers described herein comprise peptides having amino acid sequences corresponding to SEQ ID NO: 1 to 543. In certain aspects the biomarker is selected from human or mouse α-II-spectrin, synapsin-1/2/3, myelin basic protein, ubiquitin carboxyl-terminal esterase L1, calcium/calmodulin-dependent protein kinase II alpha, neurofilament light and medium, parkin 2, peroxiredoxin-1/4/5/6, 14-3-3-β/ε/ζ, synaptotagmin, enolase-1/2, guanine nucleotide binding protein α and β1, superoxide dismutase 2, internexin neuronal intermediate filament protein α, tyrosine-protein phosphatase non-receptor type substrate 1, macrophage migration inhibitory factor, proteolipid protein 1, and/or caspase 7.

CSP expression waves that were consistently observed include CPSs corresponding to one or more of SEQ ID NO:1 to 544 representing peptides having an AUC of greater than 0.90, relative to two time points between −1, 0, 5, 7, 10, 15, 20, 25 days relative to immunization. In certain aspects the contrast between day −1 and 0, −1 and 5, −1 and 7, −1 and 10, −1 and 15, −1 and 20, −1 and 25, 0 and 5, 0 and 7, 0 and 10, 0 and 15, 0 and 20, 0 and 25; 5 and 7, 5 and 10, 5 and 15, 5 and 20, 5 and 25, 7 and 10, 7 and 15, 7 and 20, 7 and 25, 10 and 15, 10 and 20, 10 and 25, 15 and 20, 15 and 25, or 20 and 25 results in an AUC of about or at least 0.90, 0.91, 0.92, 0.93, 0.94, 0.95, 0.96, 0.97, 0.98, 0.99, or 1.00.

### II. ASSAYS

In certain aspects, the biomarkers of this invention can be identified, measured or detected using microwave and magnetic (M²) proteomics for quantitative liquid chromatography-tandem mass spectrometry (LC/MS/MS) and/or immunoassay.

Microwave and magnetic (M²) proteomics for quantitative liquid chromatography-tandem mass spectrometry (LC/MS/MS) can be used to assess relatively large numbers of CSPs and brain tissue specimens in murine EAE (Raphael et al., Electrophoresis 33, 3810-3819, 2012; Mahesula et al., Electrophoresis 33, 3820-3829, 2012). M² proteomics synergizes off-line microwave-assisted chemical modification of CSPs bound to magnetic C8 microparticles, multiplexed isobaric encoding, and automated sample preparation with 96-well plates. M² proteomics is also amino acid sequence- and post-translational modification-specific (Ottens et al., Mass spectrometry reviews 25, 380-408, 2006; Ottens et al., Analytical chemistry 77, 4836-4845, 2005; Haskins et al., Journal of neurotrauma 22, 629-644, 2005; Wang et al. International review of neurobiology 61, 215-240, 2004). Despite its advantages, LC/MS/MS-based proteomics of low abundance CSPs can be confounded by masking effects due to high abundance proteins, particularly in CSF and serum where protein abundance can span up to 12 orders of magnitude (Kushnir et al., Clinical chemistry 59, 982-990, 2013; Lehmann et al., Clinical chemistry and laboratory medicine: CCLM/FESCC 51, 919-935, 2013).

Microwave and Magnetic (M2) Sample Preparation. Isobaric labeling is a mass spectrometry strategy used in quantitative proteomics. Peptides or proteins are labeled with various chemical groups that are (at least nominally) isobaric, or the same in mass, but which fragment during tandem mass spectrometry to yield reporter ions of different mass. In a typical bottom-up proteomics workflow, proteins are enzymatically digested by a protease to produce peptides, which are then labeled with different isobaric tags. The samples are mixed in equal ratios. During a liquid chromatography-mass spectrometry analysis, the peptides are fragmented to produce sequence-specific product ions, which help to determine the peptide sequence, as well as the reporter tags, whose abundances reflect the relative ratio of the peptide in the samples that were combined. In certain aspects C8 magnetic beads (BcMg, Bioclone Inc.) are suspended and washed with an equilibration buffer (e.g., 200 mM NaCl, 0.1% trifluoroacetic acid (TFA)). Whole cell protein lysate (25-100 μg at 1 μg/μL) is mixed with pre-equilibrated beads and ⅓^{rd} sample binding buffer (e.g., 800 mM NaCl, 0.4% TFA) by volume. The mixture is incubated at room temperature and the supernatant removed. The beads are washed (e.g., with 40 mM triethylammonium bicarbonate (TEAB)), and then 10 mM dithiolthreitol (DTT) is added. The bead-lysate mixture is heated by microwave for 10 s. DTT is removed and iodoacetamide (IAA) is added, followed by a second microwave heating for 10 s. The beads are washed twice and re-suspended in TEAB. In vitro proteolysis is performed with trypsin and microwave heated for 20 s in triplicate. The supernatant is used immediately or stored at −80° C. Released tryptic peptides from digested protein lysates, including any reference material, are modified at the N-terminus and at lysine residues with the tandem mass tagging (TMT)-6plex isobaric labeling reagents (Thermo scientific, San Jose, Calif.) or other similar reagent. Each individual specimen is encoded with one of the TMT-127-130 reagents, while reference material is encoded with the TMT-126 and -131 reagents: anhydrous acetonitrile is added to TMT labeling reagent and microwave-heated for 10s. To quench the reaction, 5% hydroxylamine is added to the sample at room temperature. To normalize across all specimens, TMT-encoded cell lysates from individual specimens, labeled with the TMT-127-130 reagents, are mixed with the reference material encoded with the TMT-126 and -131 reagents in equal ratios. These sample mixtures, including all TMT-encoded specimens, were stored at −80° C. until further use.

Capillary Liquid Chromatography-Fourier-Transform-Tandem Mass Spectrometry (LC/FT/MS/MS) with Protein Database Searching. Capillary LC/FT/MS/MS can be performed. For unbiased analyses, the top 6 most abundant eluting ions were fragmented by data-dependent HCD with a mass resolution of 120,000 for MS and 15,000 for MS/MS. For isobaric TMT labeling, probability-based protein database searching of MS/MS spectra against the Trembl protein database (release 2012_dec29; 59,862 sequences) was performed with a 10-node MASCOT cluster (v. 2.3.02, Matrix Science, London, UK) with the following search criteria: peak picking with Mascot Distiller; 10 ppm precursor ion mass tolerance, 0.8 Da product ion mass tolerance, 3 missed cleavages, trypsin, carbamidomethyl cysteines as a static modification, oxidized methionines, and deamidated asparagines as variable modifications, an ion score threshold of 20 and TMT-6-plex for quantification.

Immunoassay requires biospecific capture reagents, such as antibodies, to capture the biomarkers. Antibodies can be produced by methods well known in the art, e.g., by immunizing animals with a biomarker and isolating antibodies that specifically bind the biomarker. Biomarkers can be isolated from samples based on their binding characteristics. Alternatively, if the amino acid sequence of a polypeptide biomarker is known, the polypeptide can be synthesized and used to generate antibodies.

This invention contemplates using traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### III. KITS

In another aspect, the present invention provides kits for detecting and/or measuring biomarkers described herein. In one embodiment, the kit comprises a solid support, such as a chip, a microtiter plate, a bead, or resin having a capture reagent or probe attached thereon, wherein the capture reagent or probe binds a biomarker described herein. Thus, for example, the kits of the present invention can comprise mass spectrometry probes for SELDI, such as ProteinChip® arrays.

The kit can also comprise a washing solution or instructions for making a washing solution, in which the combination of the capture reagent and the washing solution allows capture of a biomarker or biomarkers on the solid support for subsequent detection by, e.g., mass spectrometry. The kit includes one or more of: (a) a substrate for holding a biological sample isolated from a human subject suspected of having or know to have a CNS-disease; (b) an agent that specifically binds at least one biomarker selected from the peptides and/or proteins described herein; and (c) printed instructions for reacting the agent with the biological sample or a portion of the biological sample to detect the presence or amount of the at least one marker in the biological sample. In the kit, the biological sample can be CSF or blood, and the agent can be an antibody, aptamer, or other molecule that specifically binds at least one peptide and/or protein described herein. The kit can also include a detectable label such as one conjugated to the agent, or one conjugated to a substance that specifically binds to the agent (e.g., a secondary antibody).

In a further embodiment, such a kit can comprise instructions for suitable operational parameters in the form of a label or separate insert. For example, the instructions may inform a consumer about how to collect the sample, how to wash the probe or the particular biomarkers to be detected. In yet another embodiment, the kit can comprise one or more containers with capture reagents, and control or reference samples.

### IV. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Microwave and Magnetic (M2) Proteomics for CNS-Specific Protein Biomarkers of Experimental Autoimmune Encephalomyelitis and Multiple Sclerosis

A. Material and Methods

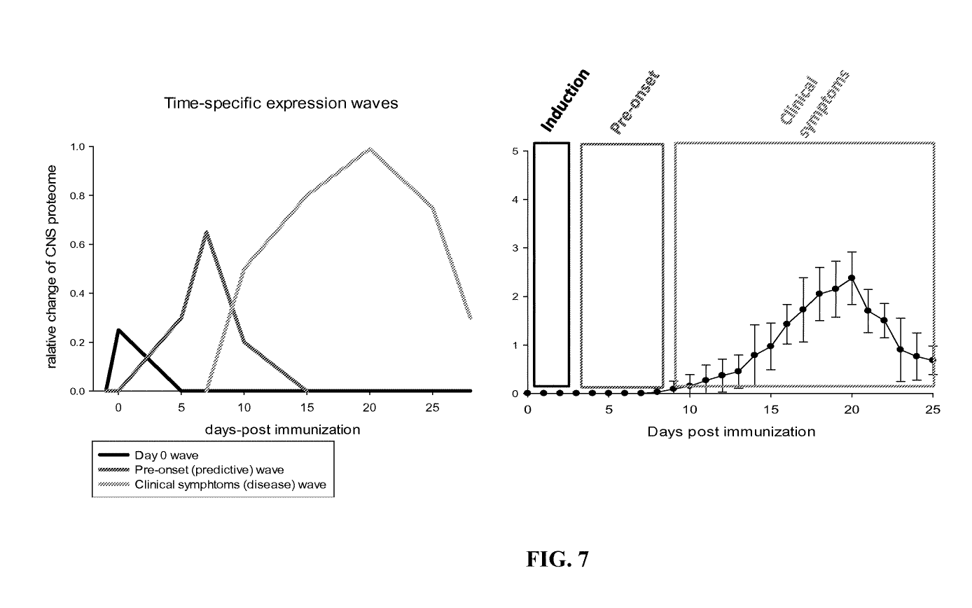

Murine Experimental Autoimmune Encephalomyelitis (EAE). Five week-old C57BL/6 female mice were purchased from the Jackson Laboratory (Stock number 000664; Bar Harbor, Me.) and used at 6-8 weeks of age in the studies. All animals were maintained in pathogen free conditions in the American Association for Laboratory Animal Science (AALAS) facility, under the guidelines established by the Institutional Animal Care and Use Committee (IACUC) at the University of Texas at San Antonio. Mice were allowed to rest for 8 days, and they were maintained under specific pathogen-free conditions. Active induction of EAE was performed with a subcutaneous injection of each mouse with 200 μg of myelin oligodendrocyte glycoprotein (MOG) 35-55 peptide (United Biochemical Research, Seattle, Wash.) in 50 μL of complete Freund's adjuvant (CFA) containing Mycobacterium tuberculosis H37Ra (Difco Laboratories, Detroit, Mich.) at a final concentration of 5 mg/mL. Two intra-peritoneal (i.p.) injections of Bordetella pertussis toxin (PTX; List Biological, Campbell, Calif.) at 200 ng per mouse were given at the time of immunization and 48 hours later (Cardali and Maugeri, Journal of neurosurgical sciences 50, 25-31, 2006). Animals were monitored and graded daily for clinical signs of EAE using the following scoring system (Hall et al., Journal of neurotrauma 22, 252-265, 2005): 0, no abnormality; 1, limp tail; 2, moderate and hind limb weakness; 3, complete hind limb paralysis; 4, quadriplegia or premoribund state; 5, death. Mice for M² proteomics were randomly selected and sacrificed at 8 disease time points, described by the number of days (d) post-immunization [−1 d (non-immunized), 0 d (3hrs post-immunization), 5 d, 7 d, 10 d, 15 d, 20 d and 25 d] (n˜20 per time point). These time points were selected to reflect inflection points of pre-onset, disease onset, early disease, disease peak, and remission (for example See FIG. 7). These time points are a practical compromise between the minimum number of mice and the minimum number of samples required to define the overall trajectory of disease progression. Brain tissue was snap-frozen in liquid nitrogen and stored at −80° C. until further use by cytokine immunoassays and M² proteomics.

Cytokine Immunoassays. Antigen-induced T cell responses were assessed in dissociated brain and spleen tissue by enzyme-linked immunosorbent spot (ELISPOT) assay for interferon-γ and interleukin-17A (IFN-γ and IL-17) as previously described (Beer et al., Journal of neurochemistry 75, 1264-1273, 2000) after stimulation with MOG35-55 peptide (United Biochemical Research, Seattle, Wash.). Briefly, ELISPOT plates (Multiscreen IP; Millipore) were coated with anti-IFN-γ (AN-18; 1 μg/mL) or anti-IL-17A (17F3; 2 μg/mL) capture antibodies in phosphate buffered saline (PBS). The plates were blocked with 1% bovine serum albumin (BSA) in PBS for 1 h at room temperature and then washed four times with PBS. After 1 hour of blocking with PBS/1% BSA, cells were added with or without antigen and incubated for 24 h at 37° C. The plates were washed three times with PBS and four times with PBS/Tween 20, and biotinylated anti-IFN-γ (R4-6A2; 0.5 μg/ml) or -IL-17A (eBioTC11-8H4; 0.125 μg/ml) detection antibodies were added and incubated overnight, respectively. Plates were washed four times with PBS/Tween 20 and incubated with streptavidin-alkaline phosphatase (Invitrogen). Cytokine spots were visualized with a BCIP/NBT phosphatase substrate (Kirkegaard & Perry Laboratories, Gaithersburg, Md.). Image analysis of ELISPOT assays was performed a Series 6 Universal-V Immunospot analyzer and Immunospot 5.1 software (Cellular Technology Limited) as described previously (Weiss et al., The Annals of thoracic surgery 88, 543-550, 2009; Jaros et al., Methods in molecular biology 1002, 1-11, 2013). Results for antigen-specific T cells were normalized with a negative control containing peptide-free media. All measurements were performed in duplicate.

Brain Tissue Lysate. Whole cell protein was extracted from brain tissue using the RIPA Lysis Buffer Kit (Santa Cruz Biotechnology, Inc. Santa Cruz, Calif.) according to the manufacturer's protocol. Briefly, an appropriate amount of RIPA complete lysis buffer was added to cell pellet. The mixture was incubated on ice for 5 minutes, followed by centrifugation at 14000×g for 15 min at 4° C. The supernatant was collected as brain tissue lysate and stored at −80° C. until further use. Protein concentration was determined using Invitrogen EZQ Protein Quantitation Kit (Invitrogen, Grand Island, N.Y.). Protein from all mice (n=157), spanning all time points, was pooled as reference material.

Microwave and Magnetic (M2) Sample Preparation. For isobaric TMT labeling, protein was pooled from all specimens by protein amount as reference material prior to sample preparation from individual specimens. Approximately 50 mg of C8 magnetic beads (BcMg, Bioclone Inc.) were suspended in 1 mL of 50% methanol. Immediately before use, 100 μL of the beads were washed 3 times with equilibration buffer (200 mM NaCl, 0.1% trifluoroacetic acid (TFA)). Whole cell protein lysate (25-100 μg at 1 μg/μL) was mixed with pre-equilibrated beads and ⅓^{rd} sample binding buffer (800 mM NaCl, 0.4% TFA) by volume. The mixture was incubated at room temperature for 5 min followed by removing the supernatant. The beads were washed twice with 150 μL of 40 mM triethylammonium bicarbonate (TEAB), and then 150 μL of 10 mM dithiolthreitol (DTT) was added. The bead-lysate mixture underwent microwave heating for 10 s. DTT was removed and 150 μL of 50 mM iodoacetamide (IAA) added, followed by a second microwave heating for 10 s. The beads were washed twice and re-suspended in 150 μL of 40 mM TEAB. In vitro proteolysis was performed with 4 μL of trypsin in a 1:25 trypsin-to-protein ratio (stock=1 μg/μL in 50mM acetic acid) and microwave heated for 20 s in triplicate. The supernatant was used immediately or stored at −80° C. Released tryptic peptides from digested protein lysates, including the reference materials described above, were modified at the N-terminus and at lysine residues with the tandem mass tagging (TMT)-6plex isobaric labeling reagents (Thermo scientific, San Jose, Calif.). Each individual specimen was encoded with one of the TMT-127-130 reagents, while reference material was encoded with the TMT-126 and -131 reagents: 41 μL of anhydrous acetonitrile was added to 0.8 mg of TMT labeling reagent for 25 μg of protein lysate and microwave-heated for 10 s. To quench the reaction, 8 μL of 5% hydroxylamine was added to the sample at room temperature. To normalize across all specimens, TMT-encoded cell lysates from individual specimens, labeled with the TMT-127-130 reagents, were mixed with the reference material encoded with the TMT-126 and -131 reagents in 1₁₂₆:1₁₂₇:1₁₂₈:1₁₂₉:1₁₃₀:1₁₃₁ ratios. These sample mixtures, including all TMT-encoded specimens, were stored at −80° C. until further use.

Capillary Liquid Chromatography-Fourier-Transform-Tandem Mass Spectrometry (LC/FT/MS/MS) with Protein Database Searching. Capillary LC/FT/MS/MS was performed with a splitless nano LC-2D pump (Eksigent, Livermore, Calif.), a 50 μm-i.d. column packed with 7 cm of 3 μm-o.d. C18 particles, and a hybrid linear ion trap-Fourier-transform tandem mass spectrometer (LTQ-ELITE; ThermoFisher, San Jose, Calif.) operated with a lock mass for calibration. The reverse-phase gradient was 2 to 62% of 0.1% formic acid (FA) in acetonitrile over 60 min at 350 nL/min. For unbiased analyses, the top 6 most abundant eluting ions were fragmented by data-dependent HCD with a mass resolution of 120,000 for MS and 15,000 for MS/MS. For isobaric TMT labeling, probability-based protein database searching of MS/MS spectra against the Trembl protein database (release 2012_dec29; 59,862 sequences) was performed with a 10-node MASCOT cluster (v. 2.3.02, Matrix Science, London, UK) with the following search criteria:peak picking with Mascot Distiller; 10 ppm precursor ion mass tolerance, 0.8 Da product ion mass tolerance, 3 missed cleavages, trypsin, carbamidomethyl cysteines as a static modification, oxidized methionines and deamidated asparagines as variable modifications, an ion score threshold of 20 and TMT-6-plex for quantification.

ELISA Immunoassays. Commercial ELISA Kits for α-II-spectrin (SEA292Mu) and synapsin-1 (SEC883Mu) were used per the manufacturer's suggested protocol (USCN Life Science Inc), where 500 μg of protein pooled from each time point (n˜20 per group) was added to the corresponding well in each plate. Plates were read at OD 450 nm for absorbance on a Synergy HT microplate reader (BioTek). For serum specimens, serum was collected from mice with mild EAE disease (disease score=1; day 10) or severe disease (score=3-4; day 20) and 1,500 μg of pooled serum samples were analyzed in triplicate with 4-5 mice per pool.

Prioritization of CSPs and Other Protein Biomarkers. CSPs and other protein biomarkers for MS patients were selected from our dataset by excluding proteins with the following descriptive terms for the protein name found in the Trembl protein database: heat shock, tubulin, histone, albumin, globin, lysosomal, mitochondrial, actin, dehydrogenase, myosin, transferrin, fructokinase, fructose, citrate, cytochrome c, glutathione, microtubule, ATP, clathrin, centromere, NADH, centrosomal, non-neuronal, elongation factor, peroxisomal, annexin, hexokinase, pyruvate, ribosomal, nucleoside, cofilin, titin, transcriptional inhibitory, initiation factor, glutamine, dynamin, RNA, cytoskeleton-associated, transducin, growth factor, vacuolar, tumor-related, phosphorylase, ribonucleoprotein, peptidyl-prolyl cis-trans isomerase, CoA, excision repair, phosphatase, zinc finger, triosephosphate isomerase, adenylyl, and keratin.

Statistical Analysis. The M2 proteomics results for each technical replicate estimate peptide expression for individual mice, encoded in sample mixtures, relative to pooled reference material from all mice, spanning all time points. Relative peptide expression levels were transformed to log base 2 for quantile normalization. Outlier arrays were removed based upon the following quality control procedures: (1) overall intensity histograms of normalized expression were compared with kernel smoothed density plots, and (2) hierarchical clustering of sample profiles was performed to assess the consistency of technical and biological variation. The association between relative peptide expression and EAE score was tested using a linear mixed-effect while treating EAE score as a continuous predictor. First, the EAE effect on relative peptide expression singly was treated as a univariate predictor. Next, the effects of EAE score were considered by adjusting for time as a quadratic effect. Changes in relative peptide expression with post-immunization time were tested using a linear mixed-effect model in which time was a treated as a multilevel factor. All the pairwise differences in relative peptide expression between all disease time points were tested, including both non-immunized mice (day −1) and 3 hrs post-immunization (day 0), using an unpaired, unequal variance t-test on the replicate averages. The relationship between the overall peptide expression profile with time or EAE score were examined using a hierarchical clustering display based upon Euclidean distance and complete linkage. For clustering analyses of relative peptide expression profiles, the subset of peptides that were most variable were considered by selecting the peptides in the top quartile (top 25%) by their standard deviation ranking Finally, the area under the receiver operating characteristic curve (AUC) were investigated for top-scoring peptides (AUC>0.9) from CSPs and other protein biomarkers, prioritized from the dataset as described above (Koutroukides et al., Journal of separation science 34, 1621-1626, 2011), and compared these values with overall p-values. Proteins were selected only if at least one peptide met the following inclusion criteria: (1) significant differential expression (AUC>0.9) between two post-immunization time points and (2) significant differential expression (overall p-value<1.0E-03) across all post-immunization time points. All statistical analysis was performed with R v3.0.2 (R-Project, Vienna, Austria).

Pathway and Network Analysis. Pathway and network analysis was performed with Ingenuity Pathways Analysis (IPA, Ingenuity R Systems) according to the manufacturer's suggestions. Briefly, MASCOT results were imported to IPA as .csv files and IPA's core analysis was performed on each file. Differentially expressed proteins corresponding to genes in the IPA knowledgebase were mapped onto canonical signaling pathways and molecular networks per the manufacturer's recommendations. A vertical bar plot, showing the percentage of proteins quantified in each canonical signaling pathway, was visualized to investigate pathway and molecular network enrichment during disease progression, where p-values for enrichment were assigned by IPA.

B. Results

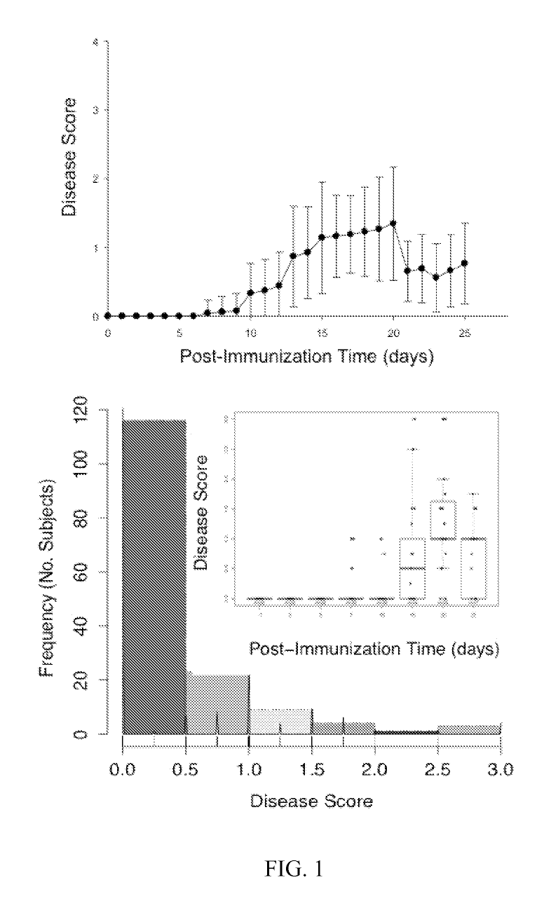

The EAE score distribution and disease trajectory for mice that were blindly scored for clinical symptoms, including those randomly selected for analysis with M2 proteomics, are shown in FIG. 1. Consistent with previous reports, only a few mice exhibited mild clinical symptoms (EAE score≦1) by day 7 (pre-onset), with increased EAE scores evident for disease onset at day 10 (EAE score≧1) and disease peak at day 20 (EAE score≧2), followed by decreased EAE scores for remission at day 25 (Shen et al., Nature 507, 366-370, 2014; Sriskantharajah et al., Journal of immunology 192, 3518-3529, 2014; Sosa et al., Journal of immunology 191, 5848-5857, 2013).

Next, expression changes in the CNS proteome were tested in order to prioritize CSPs of EAE. Focus was on analysis of the brain-tissue proteome because MS is a major target of the brain (Peterson et al., Annals of neurology 50, 389-400, 2001; Klaver et al., Prion 7, 66-75, 2013; Lassmann, Philosophical transactions of the Royal Society of London. Series B, Biological sciences 354, 1635-1640, 1999). Overall, decoding isobarically-labeled peptide expression for each specimen relative to pooled reference materials enabled statistical calculations for 1032 peptides from CSPs and other protein biomarkers (from a total of 6608 peptides and 4512 proteins) with significant differential expression between two post-immunization time points (pair-wise time point contrasts) and significant differential expression across all post-immunization time points (overall p-value). Specifically, top-scoring peptides were required to have pair-wise time point contrast with an area under the receiver operating characteristic curve (AUC) greater than 0.9 and an overall p-value of less than 1.0E-03, respectively.

M2 proteomics revealed characteristic CSP expression waves, including synapsin-1 and α-II-spectrin, which peaked at day 7 in brain tissue and preceded clinical EAE symptoms that began at day 10 and peaked at day 20.

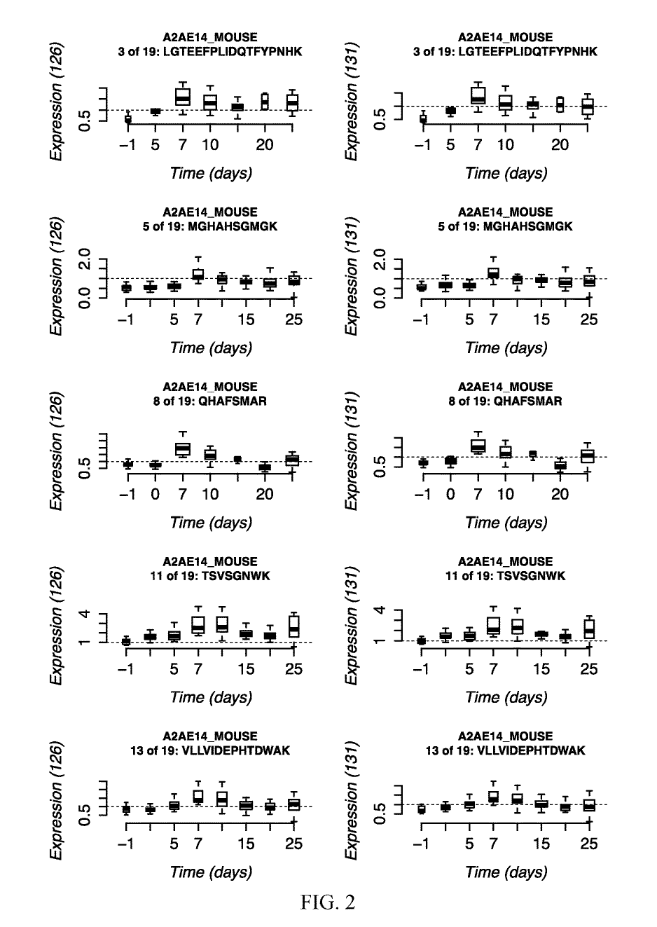

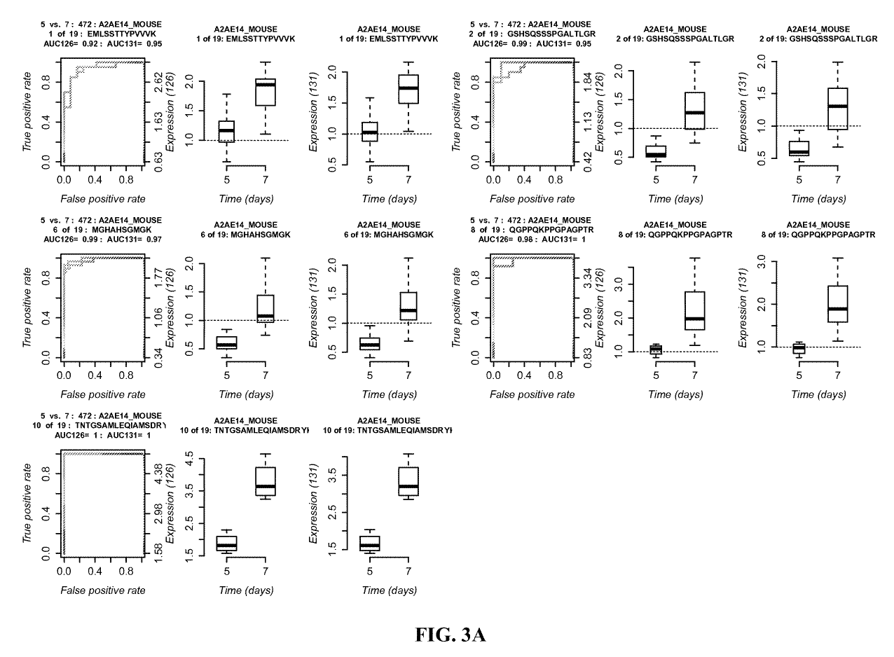

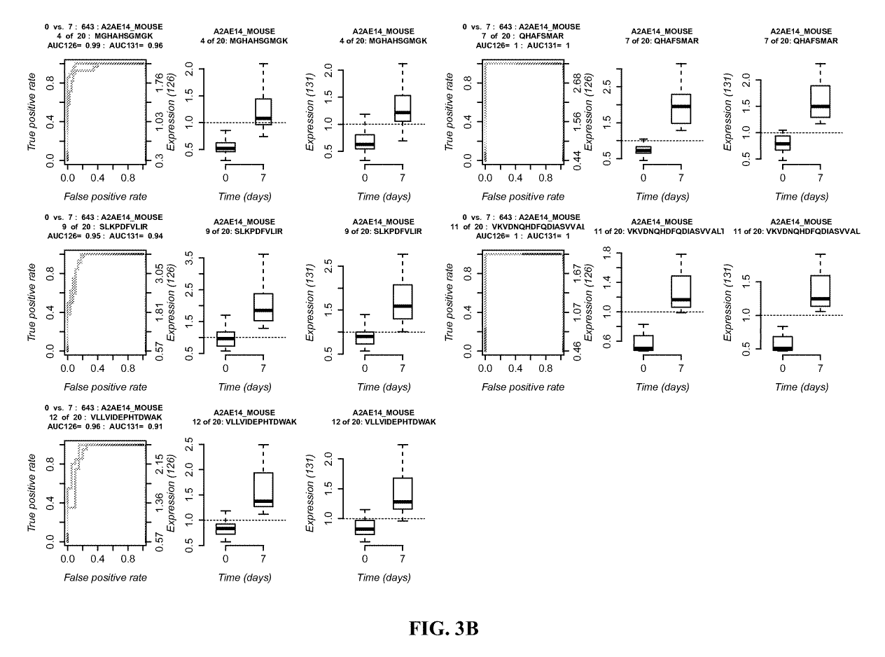

FIG. 2 shows synapsin-1 as a representative example for the expression of CSPs over the course of disease. Of the 19 peptides that were observed from synapsin-1 across all time points, 17 showed a significant differential expression with peak levels at day 7 (5 representative peptides are shown in FIG. 2). A pair-wise time point contrast from another representative peptide from synapsin-1 is shown in FIG. 3. Here, the AUC for peptide GSHSQSSSPGALTLGR (SEQ ID NO:163) for day 5 vs. day 7 was 0.99 and 0.95 for reference material tagged with the TMT126 and TMT131 isobaric labeling reagents, respectively. The overall p-values for peptide GSHSQSSSPGALTLGR (SEQ ID NO:163) were 8.1E-12 or 1.9E-08 for reference material chemically modified with the TMT126 and TMT131 isobaric labeling reagents, respectively. Correlations of relative peptide expression to post-immunization time were superior to correlations to EAE score (8.8E-01 and 4.4E-01, respectively). The remaining 2 of 19 peptides (QASISGPAPTK (SEQ ID NO:377) and QGPPQKPPGPAGPTR (SEQ ID NO:383)) were outliers, with AUC values less than 0.9 for pair-wise time point contrasts that included day 7 (e.g., day 0 vs. day 7).

CSPs and other protein biomarkers for MS patients were prioritized and selected from the dataset by excluding non-CSPs with descriptive terms for the protein name found in the Trembl protein database. Consequently, approximately four-fold more peptides (and CSPs) were observed than previously reported by applying the more stringent constraints described above (e.g., overall p-values less than 5.0E-02; AUC calculation). Statistical correlations of peptide expression to EAE score and post-immunization time were also improved and resulted in the identification of new CSP biomarkers, including: synapsin-1 and α-II-spectrin. In addition, the overall p-value previously reported for the peptide LIETYFSK (SEQ ID NO:270) from proteolipid protein 1 improved from 8.0E-04 to 7.9E-12 with the TMT126-labeled reference material. Additional confidence in this result was provided by the TMT131-labeled reference material (overall p-value=4.7E-12). Similar results were obtained for the peptide GLSATVTGGQK (SEQ ID NO:147) from proteolipid protein 1. Lastly, AUC values greater than 0.9 were observed for both peptides.

In addition to synapsin-1 and α-II-spectrin, differentially expressed CSPs and other proteins included: synapsin-2/3, myelin basic protein, ubiquitin carboxyl-terminal esterase L1, calcium/calmodulin-dependent protein kinase II alpha, neurofilament light and medium, park 2, peroxiredoxin-1/4/5/6, 14-3-3-β/ε/ζ, synaptotagmin, enolase-1/2, guanine nucleotide binding protein α and β1, superoxide dismutase 2, internexin neuronal intermediate filament protein α, tyrosine-protein phosphatase non-receptor type substrate 1, macrophage migration inhibitory factor, proteolipid protein 1, and caspase 7.

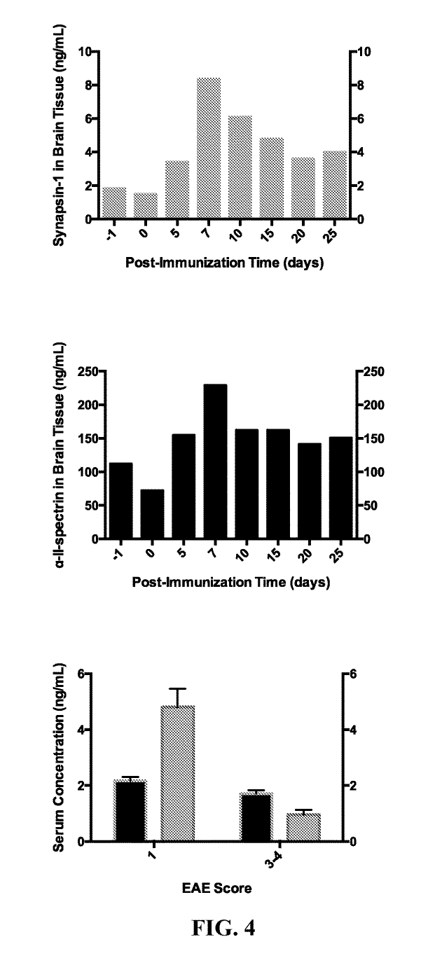

CSP expression waves, revealed with M2 proteomics of brain tissue, were confirmed with ELISAs of representative CSPs in serum specimens from an independent cohort (FIG. 4). Synapsin-1 and α-II-spectrin were selected because of their statistical significance. Serum was collected, pooled and analyzed with ELISAs from mice with EAE scores similar to the EAE scores of mice at the specific post-immunization time points that were analyzed with M2 proteomics. A strong correlation between the levels of synapsin-1 and α-II-spectrin in brain tissue and serum was observed, with peak levels at day 7 (FIG. 4).

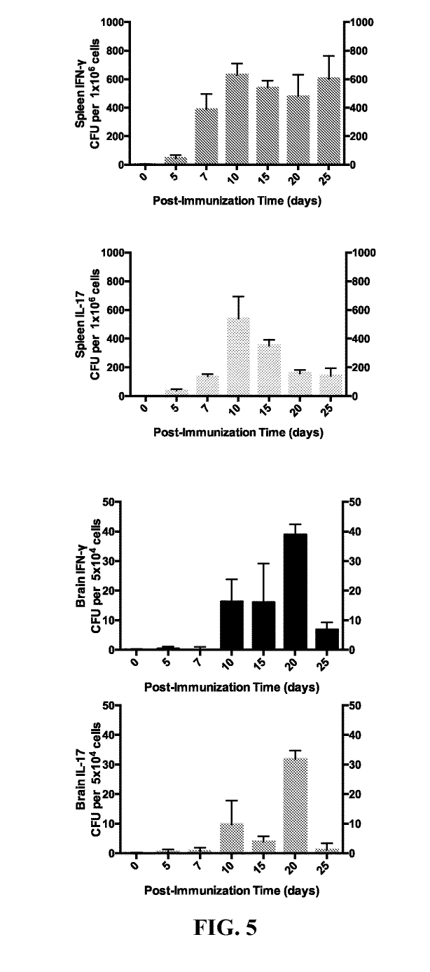

To provide a better understanding of the mechanism underlying the characteristic CSP expression waves, CNS-infiltrating inflammatory cell responses were investigated by immunoassays over the course of EAE. Cytokine ELISPOT assays showed that neuroantigen-reactive T cells producing two well-studied pathogenic cytokines (IFN-γ, and IL-17) could be detected in spleen tissue as early as day 5 after disease induction, and T cell responses peaked by day 15 (FIG. 5). In contrast, notable neuroantigen-specific T cell responses could be detected in the brain by day 10, coinciding with the onset of EAE. Consistent with previous findings, the frequencies of cytokine-producing T cells in brain tissue peaked by day 20, coinciding with peak EAE disease scores (Raphael et al., Electrophoresis 33, 3810-3819, 2012; Mahesula et al., Electrophoresis 33, 3820-3829, 2012). Importantly, the frequencies of neuroantigen-reactive T cells were negligible on day 7, consistent with the very low number of inflammatory cells detectable in the CNS at this time point (data not shown). In agreement with the notion that very few inflammatory cells were present in the CNS by day 7 after induction of disease, M2 proteomics of CD4 (a representative marker of CNS-infiltrating T cells) before day 10. Consistent with cytokine ELISPOT results, CD4 expression increased with the onset of disease at day 10 and peaked in brain tissue by day 20.

Overall, the results showed a disproportional relationship between the significant changes in CSP expression detected at day 7 and the very low number of CNS-infiltrating inflammatory cell responses at this time point. Since the magnitude of the peripheral (e.g. spleen or blood) neuroantigen-specific immune response was not correlated to EAE onset or severity, the results show that CSPs are more sensitive biomarkers for inflammatory demyelinating CNS disease than inflammatory biomarkers such as cytokines. Moreover, CSPs are also expected to be more specific biomarkers for early detection of EAE because they are not expected at appreciable levels in healthy controls, whereas neuroantigen-specific immune responses can be detected in healthy individuals (Encinas et al., Journal of immunology 157, 2186-2192, 1996; Bahmanyar et al., Journal of neuroimmunology 5, 191-196, 1983).

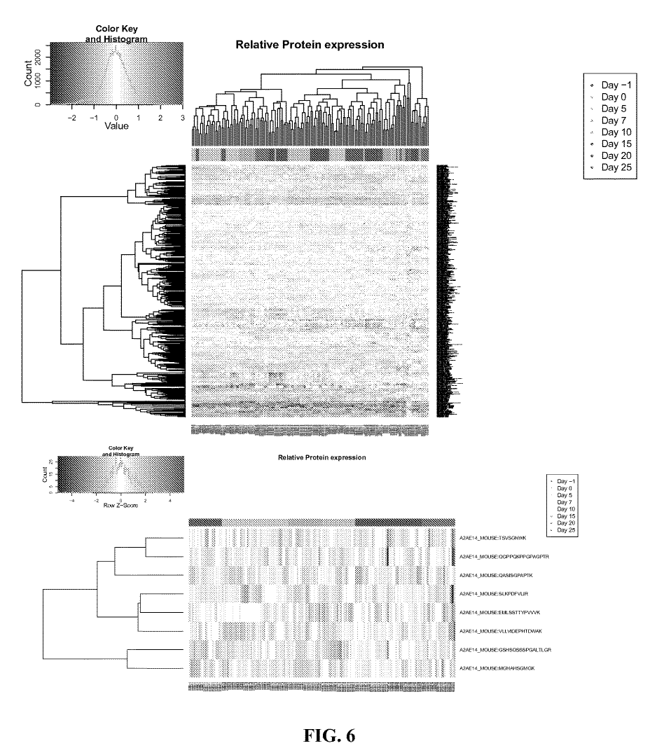

Stratification of risk groups was further investigated with non-supervised and supervised hierarchical clustering of relative peptide expression and/or post-immunization time. Non-supervised hierarchical clustering did not accurately stratify subjects by post-immunization time. However, many nearest neighbor misclassifications, where subjects were incorrectly grouped adjacent to one another rather than one group removed from one another, such misclassifications between day −1 and day 0 or day 10 and day 15, were observed across all top-ranked proteins (FIG. 6A). In contrast, supervised hierarchical clustering of all top-ranked proteins recapitulated the AUC calculations. Along these lines, supervised hierarchical clustering showed that peak levels of 17 of 19 peptides from synapsin-1 clustered at day 7 (selected peptides shown in FIG. 6B). Furthermore, visual inspection revealed the same two outliers (QASISGPAPTK (SEQ ID NO:377) and QGPPQKPPGPAGPTR (SEQ ID NO:383)) that were observed in the AUC calculations. Overall, these results suggest that M2 proteomics classifiers can be constructed to stratify risk groups based on post-immunization time, which can be used as a model for progression/regression.

Pathway and network analysis showed enrichment of differentially expressed CSPs and other proteins in specific signaling pathways and molecular networks. For example, P-values for enrichment of the top-ranked molecular network entitled “neurological disease and motor dysfunction” in the Ingenuity knowledgebase were most significant at day 7, coinciding with characteristic CSP expression waves. Enrichment of other key pathways that might be important to MS patients, such as the 14-3-3 signaling pathway, were also observed to peak at day 7.

M2 proteomics of brain tissue has been shown to be an effective means to prioritize CSP biomarkers for immunoassays in CSF and serum, by measuring changes in the brain during the disease as previously suggested (Raphael et al., Electrophoresis 33, 3810-3819, 2012; Hu et al., Proteomics 6, 4321-4334, 2006; Robinson et al., Opinion. Current opinion in immunology 15, 660-667, 2003). First, M2 proteomics of CSPs in brain tissue was shown to reveal characteristic CSP expression waves that preceded the onset of clinical EAE symptoms. Second, the CNS-infiltrating inflammatory cell response and CSP expression trajectories in serum were confirmed with cytokine ELISPOT and ELISA immunoassays, respectively, for selected CSPs found to have significant expression changes prior to clinical onset. Based on these results M2 proteomics of CSPs in brain tissue is an effective means to prioritize CSP biomarkers for immunoassays in serum and/or other body fluids (e.g., CSF).

TABLE 1
Synapsin 1 (A2AE14_MOUSE) peptides with an AUC greater than 0.9.

| Post-immunization | | | | | | |
|---|---|---|---|---|---|---|
| time contrast (e.g., | | | | | Overall p- | Overall p- |
| day 0 vs. day 7) | | Peptide Sequence | AUC126 | AUC131 | value 126 | value 131 |
| -1 | 5 | VKVDNQHDFQDIASVVALTK (SEQ ID NO: 499) | 1.00 | 1.00 | 5.0E-09 | 1.9E-13 |
| -1 | 7 | LGTEEFPLIDQTFYPNHK (SEQ ID NO: 265) | 0.98 | 0.98 | 3.6E-05 | 9.5E-04 |
| -1 | 7 | MGHAHSGMGK (SEQ ID NO: 341) | 1.00 | 0.99 | 1.6E-25 | 5.9E-22 |
| -1 | 7 | QHAFSMAR (SEQ ID NO: 384) | 1.00 | 1.00 | 1.3E-19 | 4.1E-14 |
| -1 | 7 | TSVSGNWK (SEQ ID NO: 475) | 1.00 | 0.99 | 1.7E-10 | 1.7E-09 |
| -1 | 7 | VKVDNQHDFQDIASVVALTK (SEQ ID NO: 499) | 1.00 | 1.00 | 5.0E-09 | 1.9E-13 |
| -1 | 7 | VLLVIDEPHTDWAK (SEQ ID NO: 503) | 0.98 | 0.99 | 5.9E-14 | 4.6E-13 |
| -1 | 10 | KLGTEEFPLIDQTFYPNHK (SEQ ID NO: 235) | 0.94 | 0.96 | 3.6E-04 | 1.4E-06 |
| -1 | 10 | LGTEEFPLIDQTFYPNHK (SEQ ID NO: 265) | 0.98 | 0.94 | 3.6E-05 | 9.5E-04 |
| -1 | 10 | QHAFSMAR (SEQ ID NO: 384) | 0.90 | 0.92 | 1.3E-19 | 4.1E-14 |
| -1 | 10 | TSVSGNWK (SEQ ID NO: 475) | 0.98 | 0.96 | 1.7E-10 | 1.7E-09 |
| -1 | 10 | VKVDNQHDFQDIASVVALTK (SEQ ID NO: 499) | 1.00 | 1.00 | 5.0E-09 | 1.9E-13 |
| -1 | 15 | LGTEEFPLIDQTFYPNHK (SEQ ID NO: 265) | 0.94 | 0.97 | 3.6E-05 | 9.5E-04 |
| -1 | 15 | MGHAHSGMGK (SEQ ID NO: 341) | 0.94 | 0.95 | 1.6E-25 | 5.9E-22 |
| -1 | 15 | QHAFSMAR (SEQ ID NO: 384) | 0.93 | 0.96 | 1.3E-19 | 4.1E-14 |
| -1 | 15 | TSVSGNWK (SEQ ID NO: 475) | 0.95 | 0.95 | 1.7E-10 | 1.7E-09 |
| -1 | 20 | VKVDNQHDFQDIASVVALTK (SEQ ID NO: 499) | 0.96 | 1.00 | 5.0E-09 | 1.9E-13 |
| 0 | 7 | MGHAHSGMGK (SEQ ID NO: 341) | 0.99 | 0.96 | 1.6E-25 | 5.9E-22 |
| 0 | 7 | QHAFSMAR (SEQ ID NO: 384) | 1.00 | 1.00 | 1.3E-19 | 4.1E-14 |
| 0 | 7 | SLKPDFVLIR (SEQ ID NO: 434) | 0.95 | 0.94 | 1.6E-11 | 5.0E-10 |
| 0 | 7 | VKVDNQHDFQDIASVVALTK (SEQ ID NO: 499) | 1.00 | 1.00 | 5.0E-09 | 1.9E-13 |
| 0 | 7 | VLLVIDEPHTDWAK (SEQ ID NO: 503) | 0.96 | 0.91 | 5.9E-14 | 4.6E-13 |
| 5 | 7 | EMLSSTTYPVVVK (SEQ ID NO: 91) | 0.92 | 0.95 | 1.7E-07 | 1.6E-03 |
| 5 | 7 | GSHSQSSSPGALTLGR (SEQ ID NO: 163) | 0.99 | 0.95 | 8.1E-12 | 1.9E-08 |
| 5 | 7 | MGHAHSGMGK (SEQ ID NO: 341) | 0.99 | 0.98 | 1.6E-25 | 5.9E-22 |

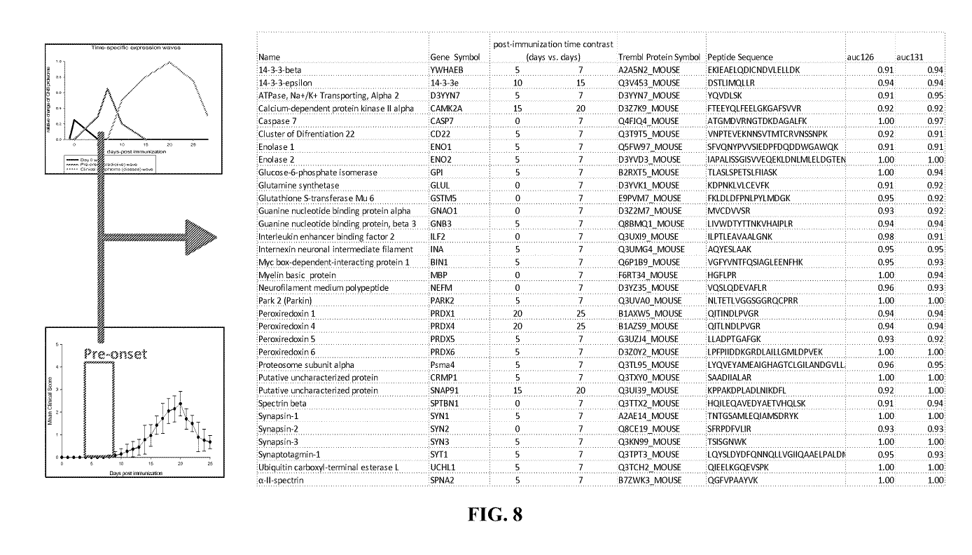

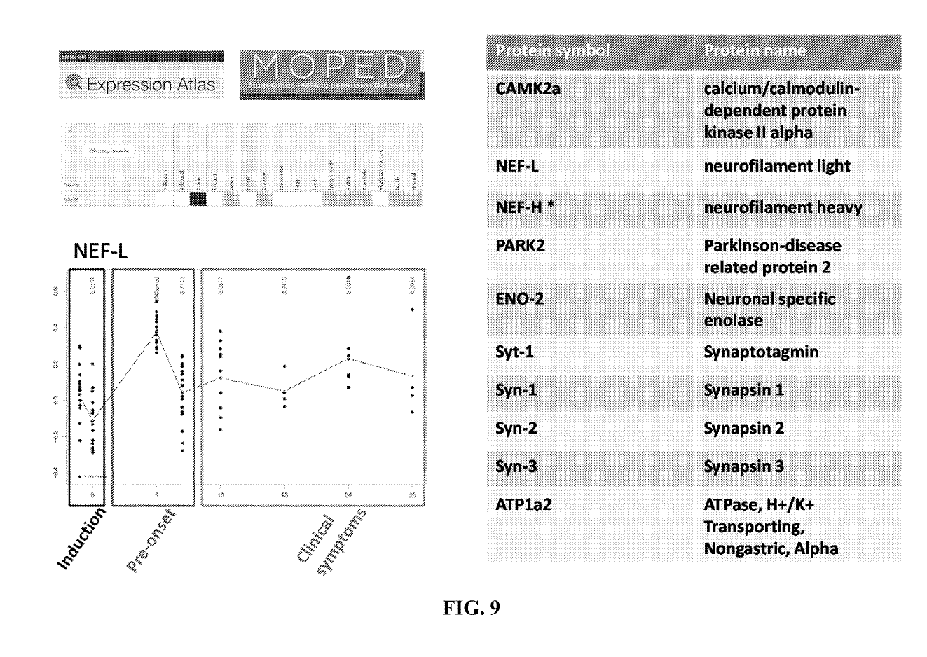

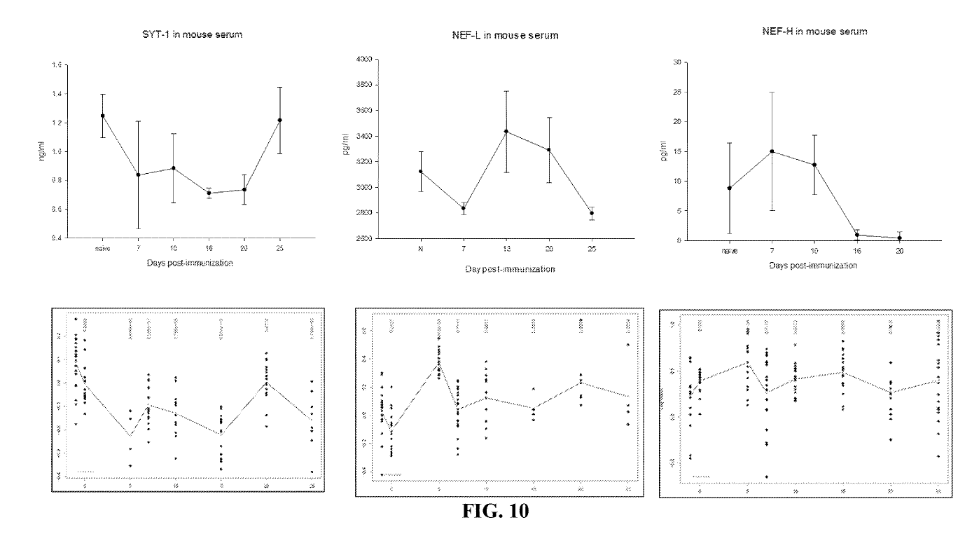

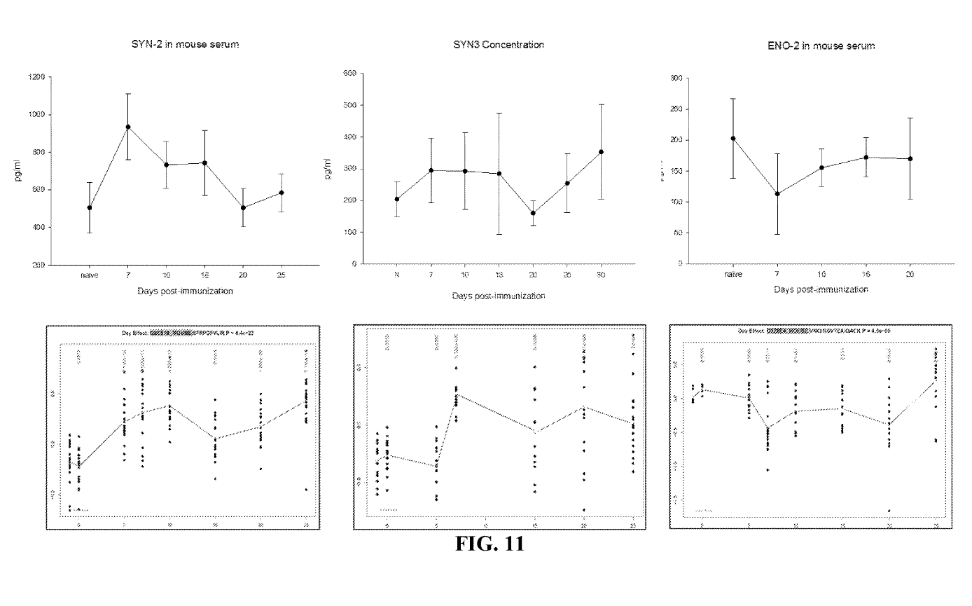

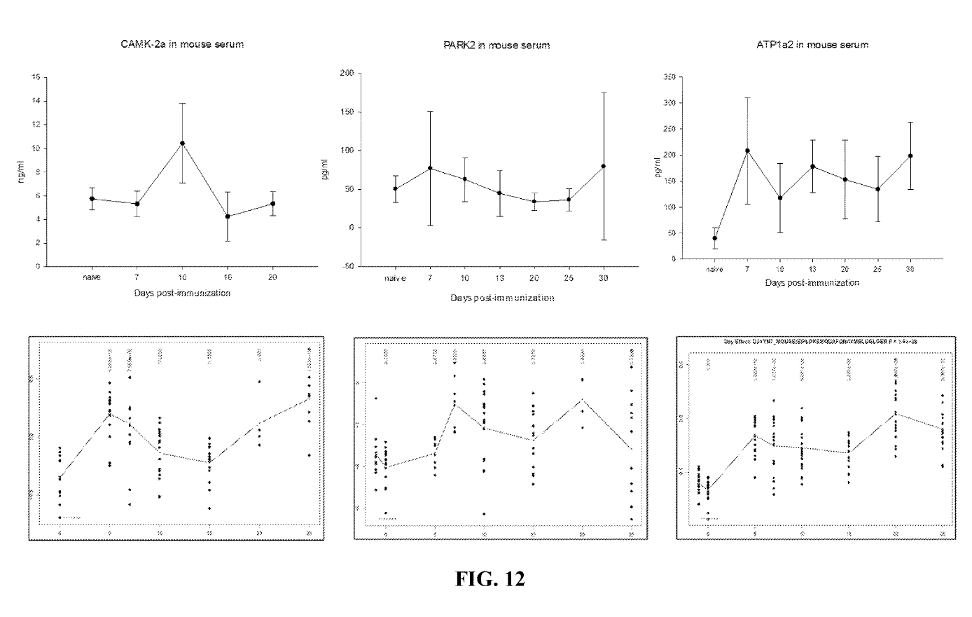

Proteomics analysis of CNS in the pre-onset phase reveals a list of biomarkers to predict disease onset. M2-proteomics identified several potential protein biomarkers in a pre-clinical model (EAE) of MS (for example see table 1 and FIG. 8). Potential biomarkers were prioritized using statistical analysis, bioinformatics tools and protein expression-trajectories. CNS-proteome analysis during the pre-onset phase of EAE revealed biomarkers which were detectable in mouse serum. Candidate predictive biomarkers for clinical-onset include: SYN-2, Syt-1, ENO-2, NEF-L, CAMK-2A (FIGS. 2, 3, 9, 10, 11, and 12). ATP1a2 may a potential diagnostic biomarker. SYN-3, PARK-2 and NEF-H did not show potential predictive values (for clinical onset) in serum.

What is claimed is:

1. A method of evaluating a patient suspected of having a central nervous system (CNS) disease comprising:
measuring the level of at least one protein biomarker or peptide biomarker selected from SEQ ID NO:1 to SEQ ID NO:544 in a biologic sample from the patient, wherein the patient is suspected of having, is at risk of developing, or has been diagnosed with a CNS disease;
classifying the subject based on the levels of at least one biomarker associated with a CNS disease or a CNS injury.

2. The method of claim 1, further comprising measuring the level of at least one protein biomarker or peptide biomarker in a plurality of samples from the patient, each sample obtained at a different time, and determining the trajectory of an expression wave for the at least one protein biomarker or peptide biomarker measured.

3. The method of claim 1, wherein the at least one protein comprises one or more of α-II-spectrin, synapsin-1/2/3, myelin basic protein, ubiquitin carboxyl-terminal esterase L1, calcium/calmodulin-dependent protein kinase II alpha, neurofilament light and medium, parkin 2, peroxiredoxin-1/4/5/6, 14-3-3-β/ε/ζ, synaptotagmin, enolase-1/2, guanine nucleotide binding protein α and β1, superoxide dismutase 2, internexin neuronal intermediate filament protein α, tyrosine-protein phosphatase non-receptor type substrate 1, macrophage migration inhibitory factor, proteolipid protein 1, or caspase 7, and combinations thereof.

4. The method of claim 1, wherein the peptide biomarker comprises one or more of a peptide having the amino acid sequence of SEQ ID NO:1 to 544.

5. The method of claim 1, wherein the protein biomarker or peptide biomarker is measured using mass spectrometry.

6. The method of claim 5, wherein the sample is labeled using isobaric labels.

7. The method of claim 6, wherein the sample is prepared using microwave and magnetic sample preparation.

8. The method of claim 1, wherein the protein biomarker or peptide biomarker is measured using an immunoassay.

9. The method of claim 8, wherein the immunoassay is an ELISA or an antibody array.

10. The method of claim 1, wherein the CNS disease is multiple sclerosis.

11. A method of detecting a preclinical expression wave comprising measuring biomarker levels in two or more samples from a patient that were obtained at different times and determining the rate of change of biomarker expression.
